(19) United States
(12) Patent Application Publication
(10) Pub. No.: US 2011/0229419 A1
(43) Pub. Date: Sep. 22, 2011

(54) CANKER SORE PREVENTION AND HEALING

(76) Inventor: Frank Lucenta, Tulsa, OK (US)

(21) Appl. No.: 12/728,084

(22) Filed: Mar. 19, 2010

(51) Int. Cl. A61K 8/19; A61Q 11/00

(52) U.S. Cl. 424/49

(57) ABSTRACT

A product for preventing and healing canker sores may comprise a mouth product; a Dead Sea salt; and sodium chloride mixture. The Dead Sea salt and the sodium chloride mixture may be mixed with the mouth product. The Dead Sea salt and sodium chloride mixture may act to prevent and heal the canker sores.

### BACKGROUND OF THE INVENTION

The present invention generally relates to preventing and healing canker sores, and more specifically adding a mixture of Dead Sea Salt and sodium chloride to mouth products such that the mixture of Dead Sea Salt and sodium chloride is deposited on the mouth, tongue, cheeks, gums, lips and throat to prevent the formation of canker sores and to heal any canker sores that are present.

Currently, consumers do not receive protection from the formation of canker sores nor do they receive effective promotion of the healing of canker sores in their mouth when using mouth products while performing activities such as brushing their teeth, rinsing their mouths or applying any other materials to their mouth, tongue, cheeks, gums, lips or throat. While current products may temporarily reduce the pain of canker sores, they do not promote healing or provide a method of prevention of canker sores.

As can be seen, what is needed are ways to prevent the formation of canker sores and promote the healing of any existing canker sores.

### SUMMARY OF THE INVENTION

In one aspect of the present invention, a product for preventing and healing canker sores may comprise a mouth product; a Dead Sea Salt; and a sodium chloride mixture. The Dead Sea Salt and the sodium chloride mixture may be mixed with the mouth product.

In another aspect of the present invention, a method for preventing or healing canker sores may comprise administering, to a user a mouth product containing an effective amount of Dead Sea Salt and sodium chloride mixture including providing a first component of the mouth product and providing a second component of the mouth product; and mixing the first component of the mouth product, the second component of the product, the Dead Sea salt, and the sodium chloride mixture to produce the mouth product.

These and other features, aspects and advantages of the present invention will become better understood with reference to the following drawings, description and claims.

### BRIEF DESCRIPTION OF THE DRAWINGS

FIGS. 1a and 1b shows flowcharts of methods for producing mouth products that prevent and heal canker sores in accordance with an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide the prevention and healing of canker sores by adding a mixture of Dead Sea Salt and sodium chloride to or mixing the Dead Sea Salt and sodium chloride mixture with mouth products regularly used by users to clean and protect their teeth and mouth, such as toothpaste, mouth rinse, mouthwash, mouth sore gel or paste, mouth spray, chewing gum, lozenges or any other products or vehicles, in order to deposit the added Dead Sea Salt and sodium chloride mixture to the mouth, tongue, cheeks, gum, lips or throat when the mouth product is used by a user. Because the Dead Sea Salt and sodium chloride mixture is water soluble, the mixture may be readily incorporated into any suitable water-based products for use in a mouth area.

While Dead Sea Salt is a unique composition containing 21 different mineral compounds known for its therapeutic, healing and anti-inflammatory properties and sodium chloride is a common compound used in many applications, such as a food preservative or a flavor enhancer, embodiments of the present invention provide a unique application for Dead Sea Salt mixed with sodium chloride to prevent and heal canker sores in and around the mouth areas of users. By adding a mixture of Dead Sea Salt and sodium chloride to products that users already regularly use in their mouth, such as toothpaste and mouthwash, the formation of canker sores may be prevented while the healing of canker sores may be promoted, thereby eliminating or relieving the pain and discomfort caused by canker sores.

The amount of the Dead Sea Salt and sodium chloride mixture to be added to mouth products may be between 0.0001% and 99.999% by volume of the final product comprising the mouth product and the Dead Sea Salt and sodium chloride mixture, and more specifically may be between 10% and 40% of the final product, and more specifically may be about 20% of the final product. Water or other acceptable carrier, diluents or adjuvant, may also be mixed or added to the Dead Sea Salt and sodium chloride mixture and the mouth products in order to provide an optimal concentration of Dead Sea salt and sodium chloride mixture within the final product.

The mixing ratio of Dead Sea salt to sodium chloride to be added to mouth products may be between 0.0001% and 99.999% by volume of the final mixture of the two, and more specifically may be between 10% and 30% of the final mixture, and more specifically may be 20% of the final mixture.

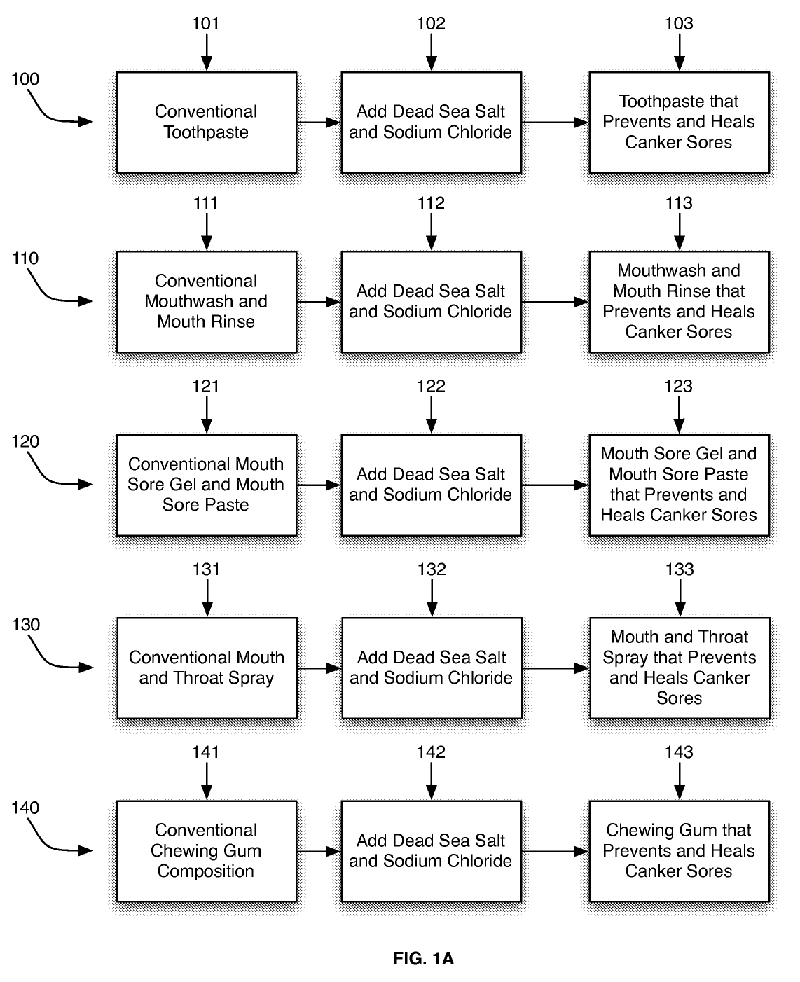

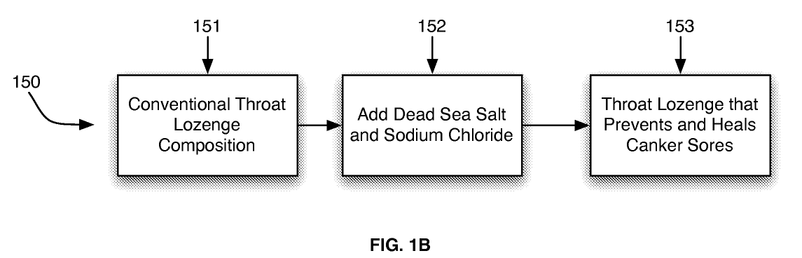

FIGS. 1a and 1b shows flowcharts of methods for producing mouth products that prevent and heal canker sores in accordance with an embodiment of the present invention. Generally, an existing mouth product may be provided, and the Dead Sea Salt and sodium chloride mixture may be added to or mixed with the existing mouth product to produce amouth product that prevents and heals canker sores. The existing mouth products may be chosen based on whether they are suitable vehicles for delivering and depositing the Dead Sea salt and sodium chloride mixture additive into the mouth of a user in an effective dose, and whether the existing mouth products should be modified to readily accept the Dead sea salt and sodium chloride mixture additive in the required effective amounts.

A flowchart 100 for a method of producing a toothpaste that prevents and heals canker sores may comprise providing an existing or modified toothpaste at 101 and adding the Dead sea salt and sodium chloride mixture to the existing toothpaste at 102, thereby producing a toothpaste that will prevent and heal canker sores at 103. The toothpaste may be a good product to use for delivering the Dead Sea salt and sodium chloride mixture to the mouth to prevent the formation of canker sores and to promote the healing of canker sores because consumers often regularly use toothpaste to brush their teeth multiple times a day. Further, by adding the Dead Sea salt and sodium chloride mixture to the toothpaste, the resulting toothpaste will include the added benefits of preventing and healing canker sores in addition to the existing benefits of the toothpaste.

A flowchart 110 for a method of producing a mouthwash or mouth rinse that prevents and heals canker sores may comprise providing an existing or modified mouthwash or mouth rinse at 111 and adding the Dead Sea salt and sodium chloride mixture to the mouthwash or mouth rinse at 112, thereby producing a mouth wash or mouth rinse that prevents and heals canker sores at 113. By adding the Dead sea salt and sodium chloride mixture to the mouthwash or mouth rinse, the resulting mouthwash or mouth rinse will include the added benefits of preventing and healing canker sores in addition to the existing benefits of the mouthwash or mouth rinse such as the prevention or reduction of bad breath, plaque formation, and gingivitis.

A flowchart 120 for a method of producing a mouth sore gel or mouth sore paste that prevents and heals canker sores may comprise providing an existing or modified mouth sore gel or mouth sore paste at 121 and adding a Dead Sea Salt and sodium chloride mixture to the mouth sore gel or mouth sore paste at 122, thereby producing a mouth sore gel or mouth sore paste that prevents and heals canker sores at 123. By adding a Dead sea Salt and sodium chloride mixture to the mouth sore gel or mouth sore paste, the resulting mouth sore gel or mouth sore paste will include the added benefits of preventing and healing canker sores in addition to the existing benefits of the mouth sore gel or mouth sore paste such as pain relief.

A flowchart 130 for a method of producing a mouth spray that prevents and heals canker sores may comprise providing an existing or modified mouth spray at 131 and adding a Dead Sea salt and sodium chloride mixture to the mouth spray at 132, thereby producing a mouth spray that prevents and heals canker sores at 133. By adding a Dead Sera salt and sodium chloride mixture to the mouth spray, the resulting mouth spray will include the added benefits of preventing and healing canker sores in addition to the existing benefits of the mouth spray.

A flowchart 140 for a method of producing a chewing gum that prevents and heals canker sores may comprise providing an existing or modified chewing gum at 141 and adding a Dead Sea Salt and sodium chloride mixture to the chewing gum at 142, thereby producing a chewing gum that prevents and heals canker sores at 143. By adding a Dead Sea salt and sodium chloride mixture to the chewing gum, the resulting chewing gum will include the added benefits of preventing and healing canker sores in addition to the existing benefits of the chewing gum.

A flowchart 150 for a method of producing a lozenge that prevents and heals canker sores may comprise providing an existing or modified lozenge at 151 and adding a Dead Sea Salt and sodium chloride mixture to the lozenge at 152, thereby producing a lozenge that prevents and heals canker sores at 153. By adding a Dead Sea salt and sodium chloride mixture to the lozenge, the resulting lozenge will include the added benefits of preventing and healing canker sores in addition to the existing benefits of the lozenge.

In accordance with alternative exemplary embodiments of the present invention, the mouth product may be instead added to a Dead sea Salt and sodium chloride mixture. Further, other products, such as water or any other suitable product may also be added to and/or mixed with the Dead Sea salt and sodium chloride mixture and the mouth product. In addition, components of a mouth product may be mixed with the Dead sea salt and sodium chloride mixture at any point in the production process of the mouth product, so that a mouth product need not to be produced first before mixing with the Dead sea Salt and sodium chloride mixture.

It should be understood, of course, that the foregoing relates to exemplary embodiments of the invention and that modifications may be made without departing from the spirit and scope of the invention as set forth in the following claims.

We claim:

1. A product for preventing and healing canker sores comprising:
a mouth product;
a Dead Sea salt; and
a sodium chloride mixture;
wherein the Dead Sea salt and the sodium chloride mixture is mixed with the mouth product.

2. The product of claim 1, wherein the mouth product comprises a toothpaste.

3. The product of claim 1, wherein the mouth product comprises a mouthwash or mouth rinse.

4. The product of claim 1, wherein the mouth product comprises a gel or paste.

5. The product of claim 1, wherein the mouth product comprises a spray.

6. The product of claim 1, wherein the product comprises a chewing gum.

7. The product of claim 1, wherein the product comprises a lozenge.

8. The product of claim 1, wherein the Dead Sea salt and sodium chloride mixture comprises between 10% to 40% of the product by volume.

9. The product of claim 1, further comprising water.

10. A method for preventing or healing canker sores, the method comprising:
administering, to a user a mouth product containing an effective amount of Dead Sea Salt and sodium chloride mixture including:
providing a first component of the mouth product and providing a second component of the mouth product; and
mixing the first component of the mouth product, the second component of the product, the Dead Sea salt, and the sodium chloride mixture to produce the mouth product.
